(19) United States
(12) Plant Patent Application Publication
(10) Pub. No.: US 2022/0304206 P1
(43) Pub. Date: Sep. 22, 2022

(54) Callistemon plant named 'CNU01'

(50) ;

(71) Applicant: Nuflora International Pty Ltd, Macquarie Fields (AU)

(72) Inventor: Graham Brown, Bowral (AU)

(73) Assignee: Nuflora International Pty Ltd, Macquarie Fields (AU)

(21) Appl. No.: 17/803,225

(22) Filed: Mar. 21, 2022

Related U.S. Application Data
(60) Provisional application No. 63/163,812, filed on Mar. 20, 2021.

(51) Int. Cl. A01H 6/00

(52) U.S. Cl. PLT/226

(57) ABSTRACT

A new and distinct variety of Callistemon plant named ‘CNU01’ which is characterized by the combination of an upright growth habit, an abundance of relatively small leaves, oblong inflorescence comprised of an abundance of white flowers, and the stability of all characteristics from generation to generation.

Latin name of the genus and species: The Latin name of the genus and species of the novel variety disclosed herein is Callistemon R. Br.

Variety denomination: The inventive variety of Callistemon disclosed herein has been given the variety denomination ‘CNU01’.

### BACKGROUND OF THE INVENTION

Parentage: ‘CNU01’ is the result of a controlled cross-pollination breeding program carried out by the inventor at a commercial plant breeding facility in Cobbitty, New South Wales, Australia in October of 2014. The inventor performed a controlled cross-pollination of the seed parent, Callistemon ‘Hot Pink’ (South African Plant Breeder's Rights grant number ZA 20043189) with Callistemon ‘Mauve Mist’ (unpatented), the pollen parent. The resulting seedlings were grown for two years to evaluate for new and distinct characteristics. In October of 2016, the inventor selected the new Callistemon plant for its compact, upright growth habit and white flowers. This new and distinctive cultivar was given the name ‘CNU01’.

Asexual Reproduction: Asexual reproduction of ‘CNU01’, by way of stem cuttings, was first initiated in 2016 in Cobbitty, New South Wales, Australia. Through greater than four subsequent generations, the unique features of this cultivar have proven to be stable and true to type.

### SUMMARY OF THE INVENTION

The cultivar ‘CNU01’ has not been observed under all possible environmental conditions and the phenotype may vary somewhat with variations in environment such as temperature, day length, and light intensity, without, however, any variance in genotype. The following traits have been repeatedly observed and are determined to be the unique characteristics of ‘CNU01’. These characteristics in combination distinguish ‘CNU01’ as a new and distinct Callistemon cultivar:
1. Callistemon ‘CNU01’ exhibits a compact, upright growth habit; and
2. Callistemon ‘CNU01’ exhibits an abundance of relatively small leaves; and
3. Callistemon ‘CNU01’ exhibits oblong, cylindrical inflorescence with an abundance of white flowers; and
4. Callistemon ‘CNU01’ exhibits flowers with white exserted stamens.

### BRIEF DESCRIPTION OF THE FIGURES

FIG. 1 illustrates, as nearly true as it is reasonably possible to make the same in color photographs of this type, an exemplary plant of ‘CNU01’. This plant is approximately 36 months old.

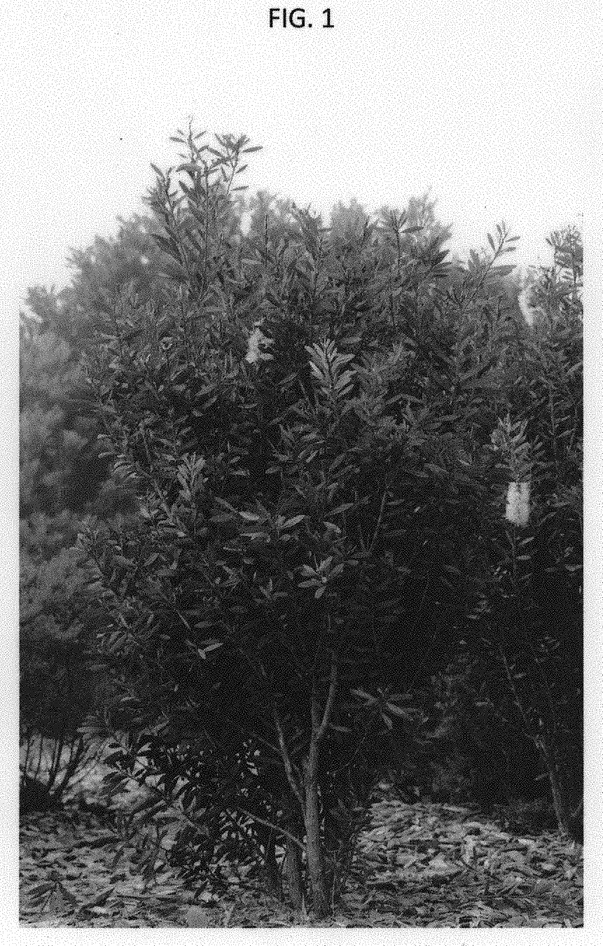

FIG. 2 illustrates, as nearly true as it is reasonably possible to make the same in color photographs of this type, the typical inflorescence of ‘CNU01’.

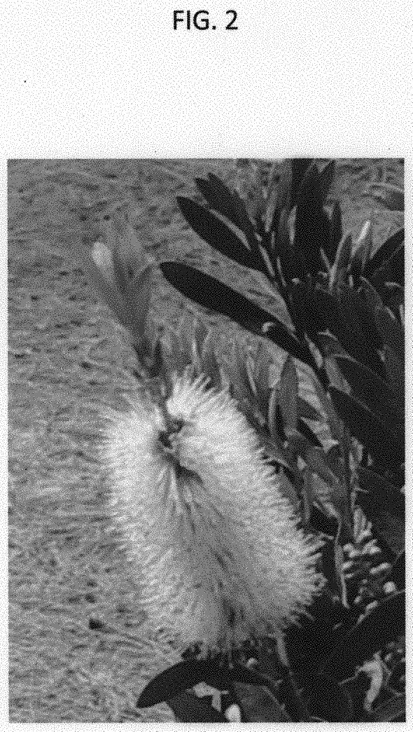

### BOTANICAL DESCRIPTION OF THE PLANT

A detailed botanical description of a new and distinct Callistemon plant known as ‘CNU01’ is not yet available. A comparison with the parents and most similar variety of common knowledge are provided below.
General plant description: Botanical description is not yet available.
Root system: Botanical description is not yet available.
Stem: Botanical description is not yet available.
Foliage: Botanical description is not yet available.
Inflorescence: Botanical description is not yet available.
Flower buds: Botanical description is not yet available.
Flower: Botanical description is not yet available.
Reproductive organs: Botanical description is not yet available.
Seed and Fruit: Botanical description is not yet available.

### COMPARISONS WITH THE PARENT PLANTS

Plants of the new cultivar ‘MACGER001’ differ from its seed parent, Callistemon ‘Hot Pink’ (South African Plant Breeder's Rights grant number ZA 20043189), by the characteristics described in Table 1.

TABLE 1

| Characteristic | ‘CNU01’ | ‘Hot Pink’ |
|---|---|---|
| General coloration | White. | Red-purple. |
| of the stamens. | | |

Plants of the new cultivar ‘CNU01’ may be distinguished from its pollen parent, Callistemon ‘Mauve Mist’ (not patented), by the characteristics described in Table 2.

TABLE 2

| Characteristic | ‘CNU01’ | ‘Mauve Mist’ |
|---|---|---|
| General coloration | White. | Purple. |
| of the stamens. | | |

### COMPARISON WITH THE MOST SIMILAR VARIETY OF COMMON KNOWLEDGE

Plants of the new cultivar ‘CNU01’ may be distinguished from the most similar known commercial comparator, Callistemon ‘White Anzac’ (unpatented), by the characteristics described in Table 3.

TABLE 3

| Characteristic | ‘CNU01’ | ‘White Anzac’ |
|---|---|---|
| Growth habit. | Upright. | Spreading. |
| Plant height. | Taller than ‘White Anzac’. | Shorter than ‘CNU01’ |
| Plant width. | Narrower than ‘White Anzac’. | Broader than ‘CNU01’. |

That which is claimed is:

1. A new and distinct variety of Callistemon plant named ‘CNU01’, substantially as described and illustrated herein.
